(19) United States
(12) Patent Application Publication
(10) Pub. No.: US 2026/0311326 A1
(43) Pub. Date: Oct. 8, 2026

(54) MACHINE LEARNING PERSONALIZED RETINAL DISEASE CARE

(71) Applicant: THE JOHNS HOPKINS UNIVERSITY, Baltimore, MD (US)

(72) Inventors: Tin Yan Alvin LIU, Baltimore, MD (US); Craig Kenneth JONES, Reisterstown, MD (US)

(21) Appl. No.: 19/481,956

(22) PCT Filed: May 6, 2024
(86) PCT No.: PCT/US2024/027926

§ 371 Date: Nov. 6, 2025

Related U.S. Application Data
(60) Provisional application No. 63/500,762, filed on May 8, 2023.

(51) Int. Cl. G06T 7/00; A61B 3/10; G16H 20/17; G16H 50/20

(52) U.S. Cl. CPC G06T 7/0012; A61B 3/102; G16H 20/17; G16H 50/20; G06T 2207/30041

(57) ABSTRACT

Machine learning techniques for providing personalized retinal disease care to a patient are presented. The techniques include: obtaining patient retinal image data and patient clinical data; providing the patient retinal image data and the patient's clinical data to a machine learning system that has been trained with training data that includes training input data and corresponding training output data, where the training data is representative of individuals at times they have not experienced a transition from dry age-related macular degeneration to wet age-related macular degeneration in either eye, where the training input data includes training retinal image data and training clinical data, and where the training output data includes training prognosis data and/or training treatment data; obtaining from the machine learning system patient output data that includes patient prognosis data and/or patient treatment data; and providing personalized retinal disease care to the patient based on the patient output data.

### CROSS-REFERENCE TO RELATED APPLICATIONS

This application is the national stage entry of International Patent Application No. PCT/US2024/027926, filed on May 6, 2024, and published as WO 2024/233432 A1 on Nov. 14, 2024, which claims the benefit of U.S. Provisional Patent Application No. 63/500,762, entitled, “MACHINE LEARNING PERSONALIZED RETINAL DISEASE CARE,” and filed May 8, 2023, which are hereby incorporated by reference herein in their entireties.

### FIELD

This disclosure relates generally to retinal disease.

### BACKGROUND

Age related macular degeneration (AMD) is a common blinding disease. In fact, AMD is the leading cause of central vision loss in people older than 50 in the US. It is estimated that 300 million people will have the disease by 2040 globally. AMD can be generally divided into a non-neovascular for (dry form AMD) and a neovascular form (wet form AMD, also known as nAMD). Most patients with AMD lose vision from the wet form; approximately 90% of vision loss in AMD is due to nAMD.

Vascular endothelial growth factor (VEGF) driven retinal diseases include, but are not limited to, AMD, diabetic eye disease, and retinal vein occlusion. Such retinal diseases can be treated with anti-VEGF injections, but such treatment has a better final outcome when the patient presents with a better visual acuity at the time of treatment commencement.

### SUMMARY

According to various embodiments, a machine learning method for providing personalized retinal disease care to a patient is presented. The method includes: obtaining patient retinal image data and patient clinical data; providing an input to a machine learning system, the input including the patient retinal image data and the patient's clinical data, where the machine learning has been trained with training data including training input data and corresponding training output data, where the training data includes data representative of individuals at times for which the individuals have not experienced a transition from dry age-related macular degeneration to wet age-related macular degeneration in either eye, where the training input data includes training retinal image data and training clinical data, and where the training output data includes at least one of training prognosis data or training treatment data; obtaining patient output data from the machine learning system, where the patient output data includes at least one of patient prognosis data or patient treatment data; and providing personalized retinal disease care to the patient based on the patient output data.

Various optional features of the above method embodiments include the following. The patient retinal image data and patient clinical data may be for a patient that has not experienced a transition from dry age-related macular degeneration to wet age-related macular degeneration in either eye. The training output data may include training treatment data, the patient output data may include patient treatment data, and the personalized retinal disease care may include treating the patient according to the patient treatment data. The patient treatment data may include a specific drug recommendation, and the treating the patient may include administering the specific drug. The specific drug recommendation may include an anti-vascular endothelial growth factor recommendation, and the treating the patient may include administering an anti-vascular endothelial growth factor injection. The method may include repeating the obtaining patient retinal image data and patient clinical data, the providing the input to the machine learning system, the obtaining patient output data, and the treating the patient. The training clinical data may include training fellow eye status data and training genetic information, where the patient clinical data includes patient fellow eye status data and patient genetic information. The machine learning system may include a first machine learning subsystem and a second machine learning subsystem, the method further including: providing the patient retinal image data to the first machine learning subsystem, where the first machine learning subsystem provides retinal image feature data; and providing the patient clinical data and the retinal image feature data to the second machine learning subsystem, where the patient output data is output from the second machine learning subsystem. The retinal image feature data may include a feature vector. The first machine learning subsystem may include a first artificial neural network or a first random forest, and the second machine learning subsystem may include a second artificial neural network or a second random forest. The training output data may include training prognosis data, the patient output data may include patient prognosis data, and the patient prognosis data may include a timeline for a transition from dry age-related macular degeneration to wet age-related macular degeneration.

According to various embodiments, a system for providing personalized retinal disease care to a patient is provided. The system includes: a non-transitory computer readable medium including instructions; and at least one electronic processor that executes the instructions to perform operations including: obtaining patient retinal image data and patient clinical data; providing an input to a machine learning system, the input including the patient retinal image data and the patient's clinical data, where the machine learning has been trained with training data including training input data and corresponding training output data, where the training data includes data representative of individuals at times for which the individuals have not experienced a transition from dry age-related macular degeneration to wet age-related macular degeneration in either eye, where the training input data includes training retinal image data and training clinical data, and where the training output data includes at least one of training prognosis data or training treatment data; obtaining patient output data from the machine learning system, where the patient output data includes at least one of patient prognosis data or patient treatment data, and where personalized retinal disease care is provided to the patient based on the patient output data.

Various optional features of the above system embodiments include the following. The patient retinal image data and patient clinical data may be for a patient that has not experienced a transition from dry age-related macular degeneration to wet age-related macular degeneration in either eye. The training output data may include training treatment data, the patient output data may include patient treatment data, and the personalized retinal disease care may include treating the patient according to the patient treatment data. The patient treatment data may include a specific drug recommendation, and the treating the patient may include administering the specific drug. The specific drug recommendation may include an anti-vascular endothelial growth factor recommendation, and the treating the patient may include administering an anti-vascular endothelial growth factor injection. The operations may further include repeating the obtaining patient retinal image data and patient clinical data, the providing the input to the machine learning system, and the obtaining patient output data. The training clinical data may include training fellow eye status data and training genetic information, and the patient clinical data may include patient fellow eye status data and patient genetic information. The machine learning system may include a first machine learning subsystem and a second machine learning subsystem, and the operations may further include: providing the patient retinal image data to the first machine learning subsystem, where the first machine learning subsystem provides retinal image feature data; and providing the patient clinical data and the retinal image feature data to the second machine learning subsystem, where the patient output data is output from the second machine learning subsystem. The retinal image feature data may include a feature vector. The first machine learning subsystem may include a first artificial neural network or a first random forest, and the second machine learning subsystem may include a second artificial neural network or a second random forest. The training output data may include training prognosis data, the patient output data may include patient prognosis data, and the patient prognosis data may include a timeline for a transition from dry age-related macular degeneration to wet age-related macular degeneration.

Combinations, (including multiple dependent combinations) of the above-described elements and those within the specification have been contemplated by the inventors and may be made, except where otherwise indicated or where contradictory.

### BRIEF DESCRIPTION OF THE DRAWINGS

Various features of the examples can be more fully appreciated, as the same become better understood with reference to the following detailed description of the examples when considered in connection with the accompanying figure, where:

FIG. 1 illustrates a machine learning system for providing personalized retinal disease care to a patient;

FIG. 2 is a diagram representing various systems based on a three-dimensional convolutional neural network that processes retinal image data only;

FIG. 3 is a diagram representing various systems based on machine learning that processes clinical data only;

FIG. 4 depicts diagrams representing various systems based on machine learning that processes both retinal image data and clinical data to provide personalized retinal disease care to a patient, according to various embodiments;

FIG. 5 summarizes the performance of the models shown and described in reference to FIGS. 2, 3, and 4; and

FIG. 6 illustrates a subgroup analysis to evaluate the performance of the systems of FIGS. 2, 3, and 4 in predicting imminent first-eye conversions.

### DESCRIPTION OF THE EXAMPLES

Reference will now be made in detail to example implementations, illustrated in the accompanying drawings. Wherever convenient, the same reference numbers will be used throughout the drawings to refer to the same or like parts. In the following description, reference is made to the accompanying drawings that form a part thereof, and in which is shown by way of illustration specific exemplary examples in which the invention may be practiced. These examples are described in sufficient detail to enable those skilled in the art to practice the invention and it is to be understood that other examples may be utilized and that changes may be made without departing from the scope of the invention. The following description is, therefore, merely exemplary.

Identification of patients at a high risk of imminent conversion (e.g., within six months) from AMD to nAMD could lead to more timely initiation of treatment and better final vision. In general, being able to identify patients at a high risk for imminent conversion to nAMD is important, as better presenting visual acuity at the time of treatment initiation with anti-VEGF injections predicts better final visual acuity.

Some embodiments use machine learning to predict, based on a combination of OCT images and clinical variables, imminent (within six months) conversion to nAMD for first and/or fellow eyes. The ability in predicting first eye conversions is particularly important from a public health point of view, as this could allow monitoring of patients with early and intermediate AMD at an optometry or even primary care setting, decompressing the patient back-log at ophthalmology practices and allowing ophthalmologists and retinal specialists to prioritize patients with more acute ophthalmic issues.

These and other features and advantages are shown and described herein in reference to the figures.

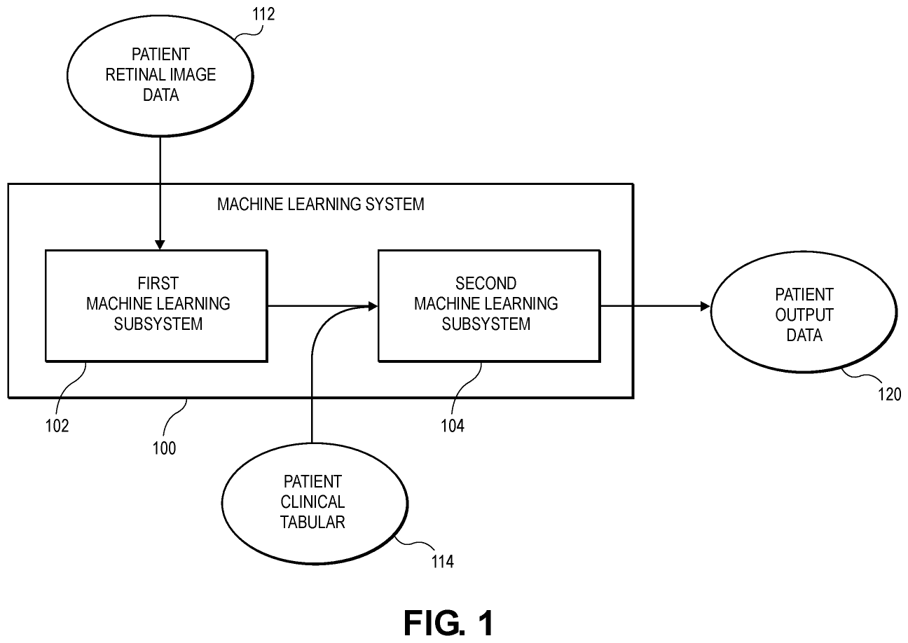

FIG. 1 illustrates a machine learning system 100 for providing personalized retinal disease care to a patient. The machine learning system 100 may be implemented in an electronic computer as disclosed herein.

The machine learning system 100 may be trained with training data, including training input data and corresponding training output data, representing a large number of (e.g., over 2000) individuals. An example non-limiting description of training the machine learning system follows.

The training input data may include multimodal data, including but not limited to training retinal image data and training clinical data. The training clinical data may include training tabular clinical data.

The training retinal image data may include, by way of non-limiting examples, optical coherence tomography (OCT) image data, angiography image data, color fundus photography image data, fundus autofluorescence image data, fluorescein angiography image data, infrared retinal image data, or indocyanine green angiography image data. According to various embodiments, structures in addition, or in the alternative, to retinas may be imaged and used for training image data. The training retinal image data may be segmented into any, or any combination, of: intraretinal hyperreflective foci, double layer sign, contour/volume/signal intensity of the retinal pigment epithelial detachment or area between the retinal pigment epithelium, and the Bruch's membrane. The segmentation may be performed manually or by a trained subsystem of the machine learning system.

The training clinical data may include, by way of non-limiting examples, any, or any combination, of: age, sex, race, visual acuity quantification, intraocular pressure quantification, fellow eye status quantification, qualitative and quantitative descriptions of examination findings, genetic information, central drusen volume, subretinal drusenoid deposits, and/or intraretinal hyperreflective foci. The training clinical data may include training tabular data.

The training output data may include training prognosis data and/or training treatment data. The training prognosis data may include, by way of non-limiting example, a timeline for a transition from dry age-related macular degeneration to wet age-related macular degeneration. The timeline may specify a predicted date for such a transition. According to some embodiments, the training prognosis data includes one or both of long term visual acuity and frequency of injections required. The training treatment data may specify, by way of non-limiting examples, any, or a combination, of: drug identification, dosage regime, and/or route of administration.

The training data thus includes a plurality, e.g., at least 2000, of data sets, where each data set represents an individual, where the training input data for the individual represents the status of the individual, and the training output data for the individual represents an outcome for the individual and/or a treatment regime provided by a medical care professional for the individual.

By way of non-limiting example, the machine learning system 100 may include multiple machine learning subsystems 102, 104. An example non-limiting description of a machine learning system that includes two machine learning subsystems forming an ensemble model, as well as its training, follows.

A first machine learning subsystem 102 may include a deep artificial neural network, a random forest model, a support vector machine, or an AdaBoost model, by way of non-limiting examples. The first machine learning subsystem 102 may be trained, e.g., using training retinal image data as described above, to receive, as an input, retinal image data, and to produce, as an output, corresponding retinal image feature data. The retinal image feature data may include, by way of non-limiting example, a patient image feature vector.

A second machine learning subsystem 104 may include a deep artificial neural network, a random forest model, a support vector machine, or an AdaBoost model, by way of non-limiting examples. The second machine learning subsystem 104 may be trained using second machine learning subsystem training input data and/or first machine learning subsystem output data. The second machine learning subsystem training input data may include training retinal image feature data, e.g., as output from the first machine learning subsystem in response to input of the training retinal image data. The second machine learning subsystem training input data may further include training clinical data as described above. The second machine learning subsystem training output data may include machine learning output data as described above, e.g., prognosis data and/or treatment data. The prognosis data may include, by way of non-limiting example, a timeline (e.g., a predicted date or amount of time) for a transition from dry age-related macular degeneration to wet age-related macular degeneration. The treatment data may specify, by way of non-limiting examples, any, or a combination, of: drug identification, dosage regime, and/or route of administration.

Once trained, the machine learning system 100 can be used to output retinal disease care data regarding a patient. A description of using a trained machine learning system to output patient retinal disease care data follows.

Once trained, the machine learning system 100 may accept patient input data 112, 114 representing a status of a patient as an input and produce, as an output, patient retinal disease care data representing a predicted prognosis for the patient and/or a recommended treatment regime for the patient.

The patient input data can include patient retinal image data 112 and patient clinical data 114 (e.g., patient clinical tabular data).

The patient image data 112 may include, by way of non-limiting examples, optical coherence tomography (OCT) image data, angiography image data, color fundus photography image data, fundus autofluorescence image data, fluorescein angiography image data, infrared retinal image data, or indocyanine green angiography image data. According to various embodiments, structures in addition, or in the alternative, to retinas may be imaged and used for patient image data. The patient image data 112 may be segmented into any, or any combination, of: intraretinal hyperreflective foci, double layer sign, contour/volume/signal intensity of the retinal pigment epithelial detachment or area between the retinal pigment epithelium, and the Bruch's membrane. The segmentation may be performed manually or by a trained subsystem of the machine learning system.

The patient clinical data 114 may include, by way of non-limiting examples, any, or any combination, of: age, sex, race, visual acuity quantification, intraocular pressure quantification, fellow eye status quantification, genetic information, central drusen volume, subretinal drusenoid deposits, and/or intraretinal hyperreflective foci. The patient clinical data 114 may include patient clinical tabular data.

The patient output data 120 may include patient prognosis data and/or patient treatment data. The patient prognosis data may include, by way of non-limiting examples, a timeline for a transition from dry age-related macular degeneration to wet age-related macular degeneration. The timeline may specify a predicted date or amount of time for such a transition. According to some embodiments, the patient prognosis data includes one or both of long term visual acuity and frequency of injections required. The patient treatment data may specify, by way of non-limiting examples, any, or a combination, of: drug identification, dosage regime, and/or route of administration.

For embodiments in which the machine learning system 100 includes two machine learning subsystems, e.g., as shown in FIG. 1, the machine learning system 100 may output patient retinal disease care data as follows. The machine learning system may accept patient input data 112, 114. The patient input data can include patient retinal image data 112 and patient clinical data 114 as described above.

The patient retinal image data 112 may be passed to the first machine learning subsystem 102 as an input. The first machine learning subsystem 102 may output corresponding patient retinal image feature data, e.g., a feature vector. The patient retinal image feature vector data may be passed to the second machine learning subsystem 104, along with patient clinical data 114, as an input. The second machine learning subsystem 104 may provide patient output data 120, as described above, as an output.

According to some embodiments, time series data may be used to train the machine learning system 100 to output patient retinal disease care data. In general, a time series may represent a sequence of data and associated times regarding each datum in the sequence of data. According to such embodiments, a time series of training data may replace single training data. According to such embodiments, once trained, the machine learning system 100 may accept a time series of patient input data. Such embodiments may output corresponding patient treatment data and/or corresponding patient prognosis data.

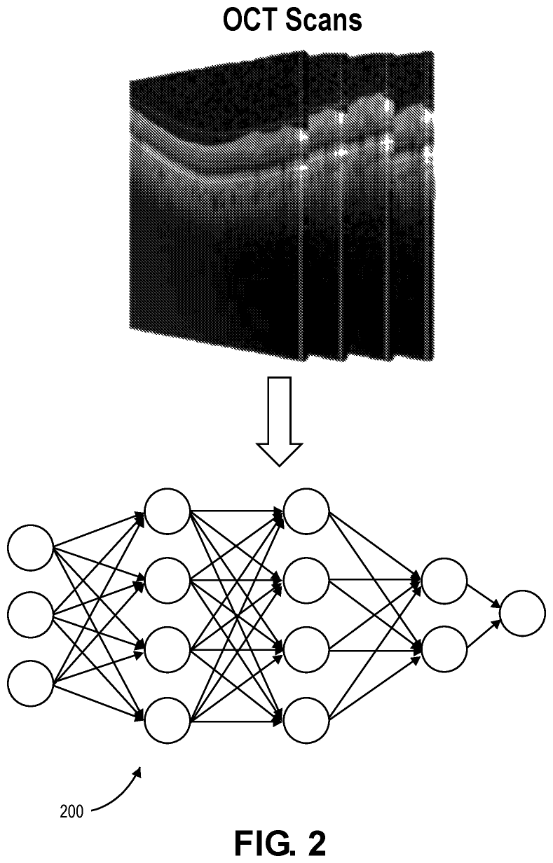

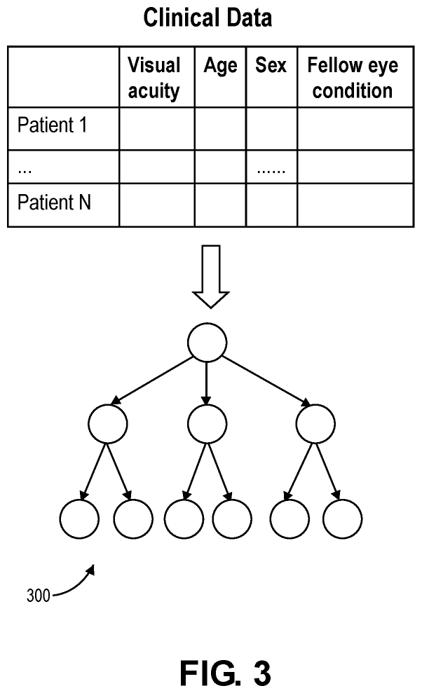

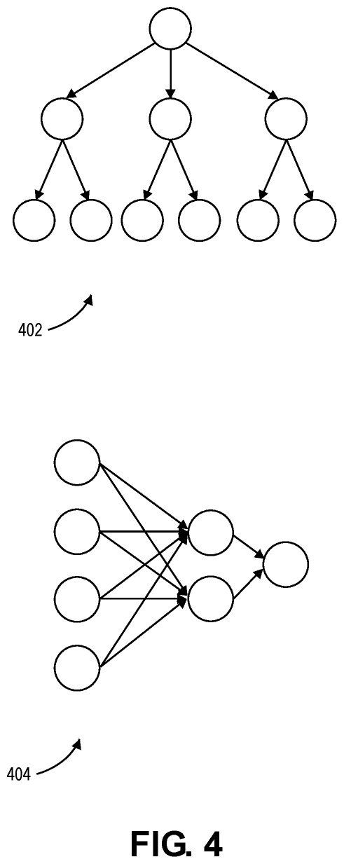

A description of various non-limiting examples of embodiments, which utilize both retinal image data and clinical data, follows. For purposes of comparison, a system that utilizes only retinal images (FIG. 2), and a system that utilizes only clinical data (FIG. 3), are also described. In particular, these systems are described and their performance compared with the described embodiments (FIG. 4). Note that, as described herein in detail, the system that utilizes only retinal image data (FIG. 2) can form a subsystem of an embodiment; for example, such a system can form the first subsystem of an embodiment as shown and described herein in reference to FIGS. 1 and 4. In particular, a system that utilizes only retinal image data as shown and described herein in reference to FIG. 2 can be a subsystem of a system that utilizes both retinal image data and clinical data as shown and described herein in reference to FIGS. 1 and 4.

The systems shown and described herein in reference to FIGS. 2, 3, and 4 utilize data from individuals as described presently. The inclusion criteria were: conversion to nAMD in at least one eye and availability of spectral domain OCT images obtained prior to conversion. Note that the data included data from individuals from times at which the individuals had not experienced nAMD conversion in either eye. The exclusion criteria were: concurrent significant macular pathologies that were not AMD related, unavailability of OCT images prior to conversion, and OCT images that were not centered on the macula. For eyes that converted to nAMD, only OCT images obtained prior to conversion were included, and these OCT images were labeled as either “more than 6 months from conversion,” or “within 6 months of conversion.” The conversion date was specified as the “date of the first anti-VEGF injection.” For eyes that never converted to nAMD, only OCT scans obtained more than 6 months prior to the last available OCT scan were included, and these OCT scans were labeled as “more than 6 months from conversion.” In total, 33,189 OCT volumes from 2,084 individuals were used for training and evaluation. Of these individuals, 63% were female. The race distribution was 89.1% White, 4.8% Black, 2.3% Asian, 0.1% Native American or Pacific Islander, and 3.8% Other. The mean and standard deviation of age of patient at the time of conversion of the 1st eye was 78.9 (+9.3) years. In addition to OCT scans, these clinical variables corresponding to each individual OCT scan were used: age at the time of scan, best corrected visual acuity at the time of scan, sex and fellow eye status (yes vs. no nAMD). For each system shown and described herein in reference to FIGS. 2, 3, and 4, the data split at the individual level was: 75% training, 15% validation and 10% testing. All systems shown and described in reference to FIGS. 2, 3, and 4 used the same training, validation, and testing splits.

FIG. 2 is a diagram 200 representing various systems based on a three-dimensional convolutional neural network that processes retinal image data only, that is, without using clinical data. Herein, the systems are both shown and described as subsystems of various embodiments, and are also are evaluated as independent systems for providing personalized retinal disease care to a patient for purposes of illustrating the superiority of various embodiments that utilize both retinal image data and clinical data. During testing, the performance of seven 3D CNN models was considered: ResNet-18, ResNet-34, ResNet-50, ResNet-101, MobileNet, GoogleNet, and DenseNet. Using transfer learning, these models were fine tuned, with the ImageNet weights as starting points. During 5-fold cross validation and using only data in the training and validation splits, the mean and standard deviation of the area under the curve (AUC) was compared across the five folds for each model, and the CNN backbone with the most robust performance was identified, namely, ResNet-50. The ResNet-50 model was used the following parameters: balanced cross entropy loss 165 (gamma=0, alpha=0.8), Adam optimizer, learning rate of 0.001, momentum of 0.9, and 100 epochs.

FIG. 3 is a diagram 300 representing various systems based on machine learning that processes clinical data only, that is, without using retinal image data. For the system of FIG. 3, two machine learning classifiers, Random Forest and XGBoost, were trained using the following clinical data only: best corrected visual acuity (BCVA), sex, age and fellow eye status. Synthetic Minority Oversampling Technique (SMOTE) was used to address class imbalance eyes within 6 months of conversion and eyes more than 6 months away from conversion.

For the systems of FIG. 3, the Random Forest model hyperparameters were tuned using the scikit-learn package Randomized Search CV method: maximum depth, the maximum of features tested by one tree, minimum samples needed to start a new leaf node, the minimum number of samples to split a node, the number of trees to make, and if bootstrapping should be used. A randomized search with 200 iterations was performed over the grid of these parameters with 3-fold cross validation. Further tuning was performed by creating ranges around the best random fit parameters found and performing a full grid search of the resulting grid of parameters. Hyperparameter tuning for the XGBoost model was performed with a grid search of possible values (including the default) and 3-fold cross validation. The parameters tested were: the learning rate, the minimum loss reduction to split a node, the maximum depth, the minimum hessian weight needed for a child tree, and the ratio of subsamples in the training set.

FIG. 4 depicts diagrams 402, 404 representing various systems based on machine learning that processes both retinal image data and clinical data to provide personalized retinal disease care to a patient, according to various embodiments. In particular, FIG. 4 represents two frameworks: Random Forest and XG Boost, represented by the diagram 402, and a multi-layer perceptron neural network, represented by the diagram 404.

The systems shown in 402 utilized Random Forest (RF) and XG Boost machine learning frameworks in the alternative, where the 3D OCT volume classification features were extracted from the final fully connected layer (2048 features) of the ResNet-50 shown and described herein in reference to FIG. 2. The extracted image feature vector and the four clinical variables (BCVA, sex, age and fellow eye status) were then concatenated into a single feature vector. Positive cases (conversion within 6 months) were up-sampled, using SMOTE. For both the Random Forest and XGBoost models, default hyperparameters were used.

For the multi-layer perceptron (MLP) framework shown in 404, the trained 3D ResNet-50 CNN of FIG. 2 was used as a feature encoder subsystem, and the MLP was used as the prediction head. The features from the final fully connected layer were downsampled from 2048 features to 10 features. This downsampled feature vector was concatenated with corresponding clinical tabular data, and passed through the MLP. The MLP consisted of 3 linear transformation layers, a batch normalization layer, and a rectified linear unit (ReLU) non-linear activation layer to reduce the dimensionality of the feature map to perform binary classification. The pre-trained 3D ResNet-50 feature encoder was frozen during training of the MLP in order to retain the learned feature images and focus the training on the combination of the image features and clinical tabular data. The final model was trained with the following hyperparameters: Adam optimizer, a learning rate of 1×10-6, balanced cross entropy loss, and 500 epochs.

A description of the statistical analysis applied to the systems shown and described in reference to FIGS. 2, 3, and 4 follows. Final model performance after training was assessed using AUC, sensitivity, and specificity calculated for the hold-out test set. For each model at a given AUC, a sensitive mode (sensitivity level set at 80%) and a specific mode (specificity level set at 80%) are reported. The 95% confidence intervals for sensitivity and specificity were calculated using the Clopper-Pearson exact method. The 95% confidence intervals for model AUCs were calculated using the percentile method (Python3: numpy.percentile) after bootstrapping the test set with replacement 10,000 times. To compare model performance, each model was evaluated on each of the bootstrapped test sets and the resulting AUC scores were compared in a pairwise fashion, resulting in a distribution of AUC difference scores. To identify significant differences in model AUC performance, a modified paired T-Test using the method developed by Nadeau and Bengio was used. This method adjusts the calculated variance of the bootstrapped AUC differences used in the calculation of the T-statistic by a factor of “(1/n+n1/n2)” rather than the traditional “1/n” to account for the non-independence of the training and test datasets, where n is the total number of cross-validations or random resamples used (100 in this study), n1 is the number of scans in the test dataset (2,051), and n2 is the number of scans in the training dataset. For the systems of FIGS. 2, 3, and 4, the value of n was selected as a bootstrap random resample number of 100 in order to evaluate significance, rather than the 10,000 previously used to estimate confidence intervals, to allow for adequate statistical reproducibility without oversampling. A range of derived p-values based on 5 repeat tests are reported, and p<0.05 is considered to be statistically significant. All model development, testing and comparisons were performed in the Microsoft Azure ML environment, using Python 3.10.8 and PyTorch 1.12.0.

In total, six models (one as shown and described in refence to FIG. 2, two as shown and described in reference to FIG. 3, and three as shown and described in reference to FIG. 4) were trained, tested and compared: MLP multimodal (trained with both OCT and clinical data), CNN OCT (ResNet-50 trained with OCT only), RF multimodal (trained with both OCT and clinical data), RF clinical (trained with clinical data only), XG Boost multimodal (trained with both OCT and clinical data), and XG Boost clinical (trained with clinical data only). The results are summarized in Table 1. Prior to integrating information from OCT volumes with clinical data for model training (FIG. 4), a correlation analysis for these two data modalities was performed to assess if the CNN had identified features from the OCT scans that encompassed the information contained in clinical data. No significant correlations between the feature vector and clinical data was found, indicating the latter could contain additional useful information not found in the OCT volumes.

TABLE 1

| Model Name | Architecture | OCT | Clinical Variable |
|---|---|---|---|
| MLP multimodal | ResNet-50 ^{a} | Y | Y |
| CNN OCT | ResNet-50 ^{a} | Y | N |
| RF multimodal | Random forest ^{b} | Y | Y |
| RF clinical | Random forest ^{b} | N | Y |
| XGBoost multimodal | XGBoost ^{c} | Y | Y |
| XGBoost clinical | XGBoost ^{c} | N | Y |

The following conventions are used in Table 1.
Abbreviations: CNN means convolutional neural network, MLP means multi-layer perceptron, OCT means optical coherence tomography, and RF means random forest.
^{a} ResNet-50 is a deep convolutional neural network.
^{b} Random Forest is a classical machine learning model.
^{c} XGBoost is a classical machine learning model.

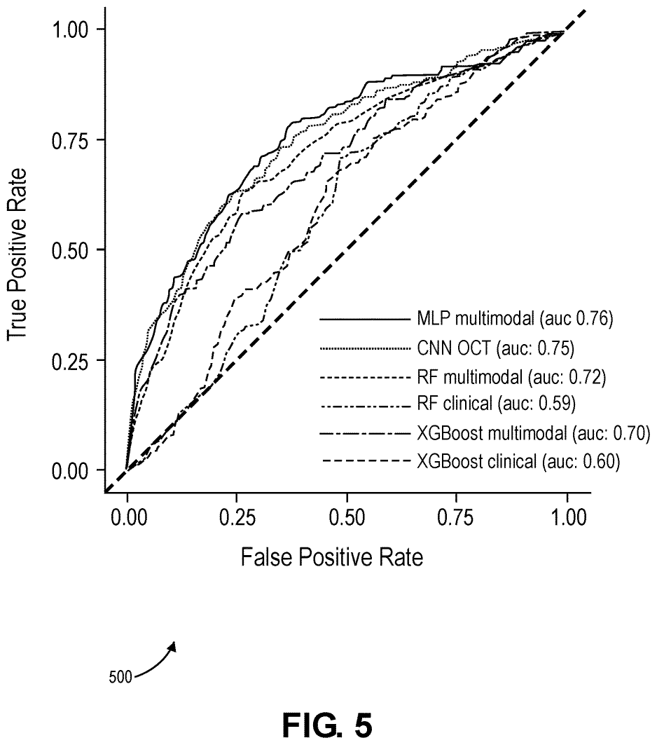

FIG. 5 summarizes the performance of the models shown and described in reference to FIGS. 2, 3, and 4. The models with the best performance were the “MLP multimodal” of FIG. 4 (AUC 0.76, 95% CI 0.71-0.80) and “CNN OCT” of FIG. 2 (AUC 0.75, 95% CI 0.70-0.79). A significant difference in AUC performance with a mean difference of 0.01 (p<0.05) was identified from paired bootstrap resampling of the test dataset, indicating that the “MLP multimodal” model generally had superior performance as compared to the “CNN OCT” model (see Table 2). The other four models, with RF and XGBoost models as backbone, generally had lower performances, with an AUC ranging from 0.70 to 0.72 when both OCT volumes and clinical data were used, and an AUC ranging from 0.59 to 0.60 when only clinical data was used.

TABLE 2

| | | | CNN OCT − | |
|---|---|---|---|---|
| | CNN | MLP | MLP Multimodal | |
| | OCT | multimodal | Mean AUC Difference | P-values |
| All-eye | 0.749 | 0.755 | +0.006 ^{b} | 0.008-0.048 * |
| conversion | (0.704-0.792) ^{a} | (0.710-0.797) ^{a} | | |
| First-eye | 0.714 | 0.730 | +0.016 ^{b} | <0.001 * |
| conversion | (0.639-0.781) ^{a} | (0.657-0.795) ^{a} | | |

The following conventions are used in Table 2.
Abbreciations: AUC means area under the curve, CNN means convolutional neural network, MLP means multi-layer perceptron, and OCT means optical coherence tomography.
^{a} 95% CI calculated from 10,000 bootstrapped samples of the test set shown in parentheses.
^{b} Significant differences between the “CNN OCT” and “MLP multimodal” models were identified using 5 repetitions of 100 bootstrapped samples of the test set, with AUC differences evaluated by the modified paired T-test.
* = statistically significant.

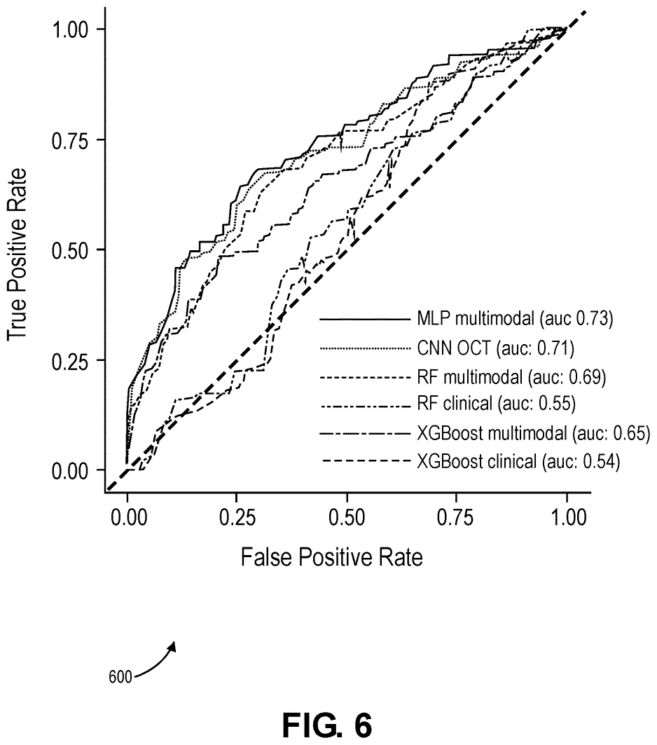

FIG. 6 illustrates a subgroup analysis to evaluate the performance of the systems of FIGS. 2, 3, and 4 in predicting imminent first-eye conversions. A general slight decrease in performance was observed in all six models compared to performance on prediction all-eye conversions. For predicting first-eye conversions, the “MLP multimodal” model of FIG. 4 had a calculated AUC of 0.73 (95% CI: 0.66-0.80), and the “CNN OCT” model of FIG. 2 had a calculated AUC of 0.71 (95% CI: 0.64-0.78). A significant difference in AUC performance with a mean difference of 0.02 (p<0.001) was identified from paired bootstrap resampling of the test dataset, indicating that the “MLP multimodal” model was superior to the “CNN OCT” model in predicting imminent first-eye conversions as well, as summarized in Table 2.

Detailed sensitivity and specificity analyses for the top two performing models, MLP multimodal and CNN OCT, are presented herein. The performance of these two models, when set to a sensitive (80% sensitive) and specific (80% specific) operating point, is summarized in Table 3.

TABLE 3

| | | (A) CNN OCT | | (B) MLP Multimodal | |
|---|---|---|---|---|---|
| | | Sensitivity | Specificity | Sensitivity | Specificity |
| All-eye | Sensitive | 79.7% | 54.1% | 79.7% | 60.4% |
| conversion | Threshold | (72.5-85.8) | (51.9-56.4) | (72.-85.8) | (58.1-62.6) |
| | Specific | 57.5% | 80.0% | 56.9% | 80.0% |
| | Threshold | (49.3-65.5) | (78.1-81.8) | (48.6-64.8) | (78.1-81.8) |
| First-eye | Sensitive | 80.2% | 43.7% | 80.2% | 45.3% |
| conversion | Threshold | (69.9-88.3) | (36.5-51.1) | (69.9-88.3) | (38.0-52.6) |
| | Specific | 51.9% | 80.0% | 53.1% | 80.0% |
| | Threshold | (40.5-63.1) | (73.6-85.4) | (41.7-64.3) | (73.6-85.4) |

The following conventions are used in Table 3.
Abbreviations: AUC means area under the curve, CNN means convolutional neural network, MLP means multi-layer perceptron, and OCT means optical coherence tomography.

Thus, the best performing model from among those shown and described in reference to FIGS. 2, 3, and 4, which was DL-based and used both OCT images and clinical tabular data for training (MLP multimodal), showed a reasonably robust performance in predicting imminent conversion to nAMD within 6 months, with an AUC of 0.76. When compared to the “CNN OCT” model, which only used OCT images for training and predictions, the “MLP multimodal” model showed statistically-significant, superior performance in predicting both all-eye and first-eye conversions. Training the “MLP multimodal” specifically included the clinical variables of age, sex, BCVA and fellow eye status.

Thus, some embodiments incorporate relevant clinical variables into the training of an OCT-based deep learning model, which is capable of accurately predicting imminent first and fellow eye conversion to nAMD within 6 months.

Note that, in contrast with, for example, Banerjee I, de Sisternes L, Hallak J A, et al., Prediction of age-related macular degeneration disease using a sequential deep learning approach on longitudinal SD-OCT imaging biomarkers, Sci Rep. Sep. 22 2020; 10(1):15434 and Yim, J., Chopra, R., Spitz, T., et al., Predicting conversion to wet age-related macular degeneration using deep learning, Nat Med 26, 892-899 (2020), which predict fellow eye nAMD conversion only after a first eye has converted, some embodiments predict first eye conversions, which is valuable from a public health perspective. Patients with nAMD in one eye already typically have to undergo regular clinic visits for intravitreal anti-VEGF injections, and therefore, it is already relatively convenient and easy to monitor the status of the fellow eye. As such, the ability to predict imminent conversion in the fellow eye will not drastically alter current practice pattern. In contrast, embodiments that can predict imminent conversion in the first eye of patients who do not have a prior history of nAMD could change current practice pattern in terms of screening, monitoring, and referral. For example, such some embodiments may be deployed as a clinical decision support tool at primary care clinics to monitor patients with early or intermediate AMD, and these patients will only be referred to a higher level of care if deemed to be at a high risk for imminent conversion. Thus, some embodiments could, in turn, allow ophthalmologists and retinal specialists to manage and prioritize patients with more acute ophthalmic issues.

Note that embodiments are not limited to age-related macular degeneration. Embodiments may be trained according to, and be used to output patient retinal disease care data, regarding any vascular endothelial growth factor (VEGF) driven retinal disease, including, for example, age-related macular degeneration, diabetic eye disease (e.g., diabetic retinopathy), and/or retinal vein occlusion. In general, embodiments may be trained according to, and be used to output patient retinal disease care data, for any retinal vascular disease.

Combinations, (including multiple dependent combinations) of the above-described elements and those within the specification have been contemplated by the inventors and may be made, except where otherwise indicated or where contradictory.

Certain examples can be performed using a computer program or set of programs. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), and magnetic or optical disks or tapes.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented using computer readable program instructions that are executed by a processor.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

In embodiments, the computer readable program instructions may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Python, Smalltalk, C++, or the like, and procedural programming languages, such as the C programming language or similar programming languages. The computer readable program instructions may execute entirely on a user's computer, partly on the user's computer, as a stand-alone software package, partly on the user's computer and partly on a remote computer or entirely on the remote computer or server.

As used herein, the terms “A or B” and “A and/or B” are intended to encompass A, B, or {A and B}. Further, the terms “A, B, or C” and “A, B, and/or C” are intended to encompass single items, pairs of items, or all items, that is, all of: A, B, C, {A and B}, {A and C}, {B and C}, and {A and B and C}. The term “or” as used herein means “and/or.”

As used herein, language such as “at least one of X, Y, and Z,” “at least one of X, Y, or Z,” “at least one or more of X, Y, and Z,” “at least one or more of X, Y, or Z,” “at least one or more of X, Y, and/or Z,” or “at least one of X, Y, and/or Z,” is intended to be inclusive of both a single item (e.g., just X, or just Y, or just Z) and multiple items (e.g., {X and Y}, {X and Z}, {Y and Z}, or {X, Y, and Z}). The phrase “at least one of” and similar phrases are not intended to convey a requirement that each possible item must be present, although each possible item may be present.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. § 112(f). However, for any claims containing elements designated in any other manner, it is intended that such elements are not to be interpreted under 35 U.S.C. § 112(f).

While the invention has been described with reference to the exemplary examples thereof, those skilled in the art will be able to make various modifications to the described examples without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

What is claimed is:

1. A machine learning method for providing personalized retinal disease care to a patient, the method comprising:
obtaining patient retinal image data and patient clinical data;
providing an input to a machine learning system, the input comprising the patient retinal image data and the patient's clinical data, wherein the machine learning has been trained with training data comprising training input data and corresponding training output data, wherein the training data comprises data representative of individuals at times for which the individuals have not experienced a transition from dry age-related macular degeneration to wet age-related macular degeneration in either eye, wherein the training input data comprises training retinal image data and training clinical data, and wherein the training output data comprises at least one of training prognosis data or training treatment data;
obtaining patient output data from the machine learning system, wherein the patient output data comprises at least one of patient prognosis data or patient treatment data; and
providing personalized retinal disease care to the patient based on the patient output data.

2. The method of claim 1, wherein the patient retinal image data and patient clinical data is for a patient that has not experienced a transition from dry age-related macular degeneration to wet age-related macular degeneration in either eye.

3. The method of claim 1, wherein the training output data comprises training treatment data, wherein the patient output data comprises patient treatment data, and wherein the personalized retinal disease care comprises treating the patient according to the patient treatment data.

4. The method of claim 3, wherein the patient treatment data comprises a specific drug recommendation, and wherein the treating the patient comprises administering the specific drug.

5. The method of claim 4, wherein the specific drug recommendation comprises an anti-vascular endothelial growth factor recommendation, and wherein the treating the patient comprises administering an anti-vascular endothelial growth factor injection.

6. The method of claim 3, further comprising repeating the obtaining patient retinal image data and patient clinical data, the providing the input to the machine learning system, the obtaining patient output data, and the treating the patient.

7. The method of claim 1, wherein the training clinical data comprises training fellow eye status data and training genetic information, and wherein the patient clinical data comprises patient fellow eye status data and patient genetic information.

8. The method of claim 1, wherein the machine learning system comprises a first machine learning subsystem and a second machine learning subsystem, the method further comprising:
providing the patient retinal image data to the first machine learning subsystem, wherein the first machine learning subsystem provides retinal image feature data; and
providing the patient clinical data and the retinal image feature data to the second machine learning subsystem, wherein the patient output data is output from the second machine learning subsystem.

9. The method of claim 8, wherein the retinal image feature data comprises a feature vector.

10. The method of claim 8, wherein the first machine learning subsystem comprises a first artificial neural network or a first random forest, and wherein the second machine learning subsystem comprises a second artificial neural network or a second random forest.

11. The method of claim 1, wherein the training output data comprises training prognosis data, wherein the patient output data comprises patient prognosis data, and wherein the patient prognosis data comprises a timeline for a transition from dry age-related macular degeneration to wet age-related macular degeneration.

12. A system for providing personalized retinal disease care to a patient, the system comprising: a non-transitory computer readable medium comprising instructions; and at least one electronic processor that executes the instructions to perform operations comprising:
obtaining patient retinal image data and patient clinical data;
providing an input to a machine learning system, the input comprising the patient retinal image data and the patient's clinical data, wherein the machine learning has been trained with training data comprising training input data and corresponding training output data, wherein the training data comprises data representative of individuals at times for which the individuals have not experienced a transition from dry age-related macular degeneration to wet age-related macular degeneration in either eye, wherein the training input data comprises training retinal image data and training clinical data, and wherein the training output data comprises at least one of training prognosis data or training treatment data; and
obtaining patient output data from the machine learning system, wherein the patient output data comprises at least one of patient prognosis data or patient treatment data, and wherein personalized retinal disease care is provided to the patient based on the patient output data.

13. The system of claim 12, wherein the patient retinal image data and patient clinical data is for a patient that has not experienced a transition from dry age-related macular degeneration to wet age-related macular degeneration in either eye.

14. The system of claim 12, wherein the training output data comprises training treatment data, wherein the patient output data comprises patient treatment data, and wherein the personalized retinal disease care comprises treating the patient according to the patient treatment data.

15. The system of claim 14, wherein the patient treatment data comprises a specific drug recommendation, and wherein the treating the patient comprises administering the specific drug.

16. The system of claim 15, wherein the specific drug recommendation comprises an anti-vascular endothelial growth factor recommendation, and wherein the treating the patient comprises administering an anti-vascular endothelial growth factor injection.

17. The system of claim 14, wherein the operations further comprise repeating the obtaining patient retinal image data and patient clinical data, the providing the input to the machine learning system, and the obtaining patient output data.

18. The system of claim 12, wherein the training clinical data comprises training fellow eye status data and training genetic information, and wherein the patient clinical data comprises patient fellow eye status data and patient genetic information.

19. The system of claim 12, wherein the machine learning system comprises a first machine learning subsystem and a second machine learning subsystem, wherein the operations further comprise:
providing the patient retinal image data to the first machine learning subsystem, wherein the first machine learning subsystem provides retinal image feature data; and
providing the patient clinical data and the retinal image feature data to the second machine learning subsystem, wherein the patient output data is output from the second machine learning subsystem.

20. The system of claim 19, wherein the retinal image feature data comprises a feature vector.

21. The system of claim 19, wherein the first machine learning subsystem comprises a first artificial neural network or a first random forest, and wherein the second machine learning subsystem comprises a second artificial neural network or a second random forest.

22. The system of claim 12, wherein the training output data comprises training prognosis data, wherein the patient output data comprises patient prognosis data, and wherein the patient prognosis data comprises a timeline for a transition from dry age-related macular degeneration to wet age-related macular degeneration.
